(19) United States
(12) Plant Patent Application Publication
(10) Pub. No.: US 2009/0007305 P1
(43) Pub. Date: Jan. 1, 2009

(54) Apple tree named 'PLFOG99'

(76) Inventors: Terry Fogliani, Kirup (AU); Dianne Fogliani, Kirup (AU)

Correspondence Address: STRATTON BALLEW, 213 S 12TH AVE, YAKIMA, WA, 98902, US

(21) Appl. No.: 12/214,786

(22) Filed: Jun. 20, 2008

Related U.S. Application Data
(60) Provisional application No. 60/937,588, filed on Jun. 27, 2007.

(51) Int. Cl. A01H 5/00

(52) U.S. Cl. PLT/161

(57) ABSTRACT

‘PLFOG99’ is a new and distinct apple tree notable for its compact tree type, spur bearing habit, and early maturity as compared to ‘Cripps Pink’ and other known cultivars.

### CROSS REFERENCE TO RELATED APPLICATIONS

This application claims priority of co-pending U.S. Provisional Application Ser. No. 60/937,588 filed Jun. 27, 2007.

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica (Borkh.)

### VARIETY DENOMINATION

‘PLFOG99’

### BACKGROUND AND SUMMARY OF THE VARIETY

The new apple variety ‘PLFOG99’ originated as a chance limb mutation on a ‘Cripps Pink’ (U.S. Plant Pat. No. 7,880) apple tree located in a commercial orchard at Kirup, Western Australia in late 1999. The limb was observed to produce fruit up to two weeks earlier than ‘Cripps Pink’ but also had a significantly different compact growth habit as compared to the ‘Cripps Pink’ tree. In 2000, ten trees were propagated from the mutated limb by budding onto high vigor ‘MM109’ apple rootstock, to determine trueness to type as compared with the original limb, for fruit maturity and quality, and compact growth habit. These trees were observed to grow very differently in the field nursery compared with ‘Cripps Pink’ and other apple trees. The growth type and habit of the trees was columnar and plant growth was compact, with fruit borne on spurs. The first crop, harvested from trees in their third leaf, was very good. These initial promising observations provided the incentive to propagate an additional 100 trees over 4 years for planting from 2002 for further evaluation. Of the 400 trees planted at Kirup, three generations have been observed to be stable and true to type to date. No off-types have been observed. The new variety has been named ‘PLFOG99’.

‘PLFOG99’ is a new and distinct apple tree notable for its compact tree type, spur bearing habit, and early maturity as compared to ‘Cripps Pink.’

### BRIEF DESCRIPTION OF THE PHOTOGRAPHS

FIG. 1 shows the tree and fruit of the new variety;

FIG. 2 shows the fruit and leaves of the new variety;

FIGS. 3 and 4 show the fruit of the new variety;

FIG. 5 shows the leaves of the new variety; and

FIG. 6 shows the blossom of the new variety.

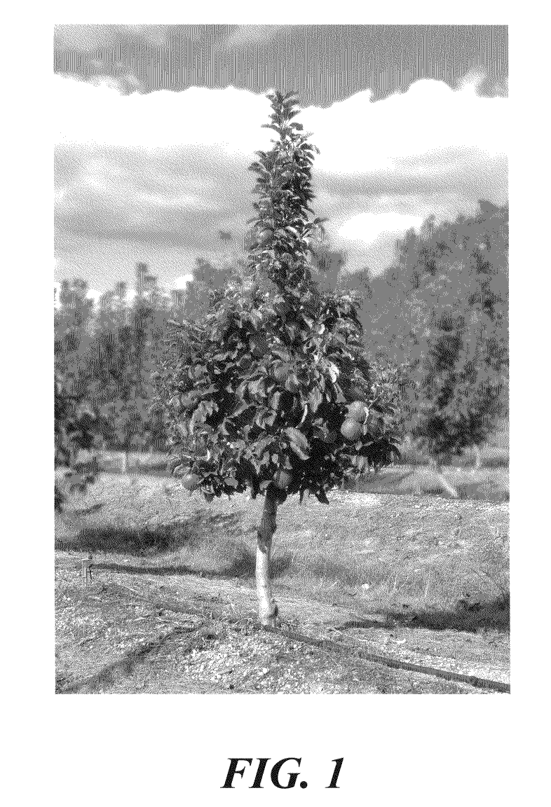

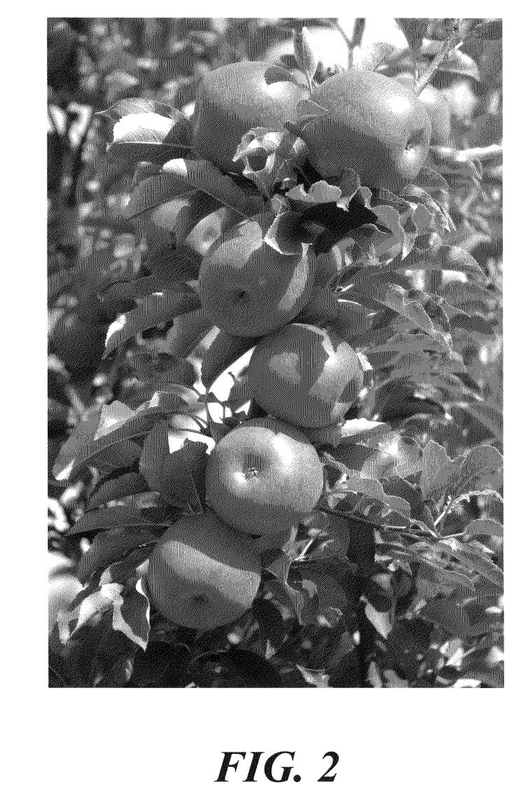

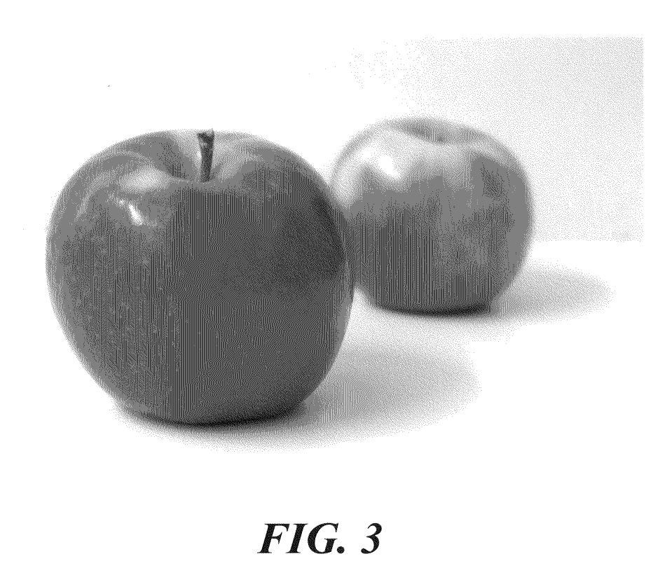

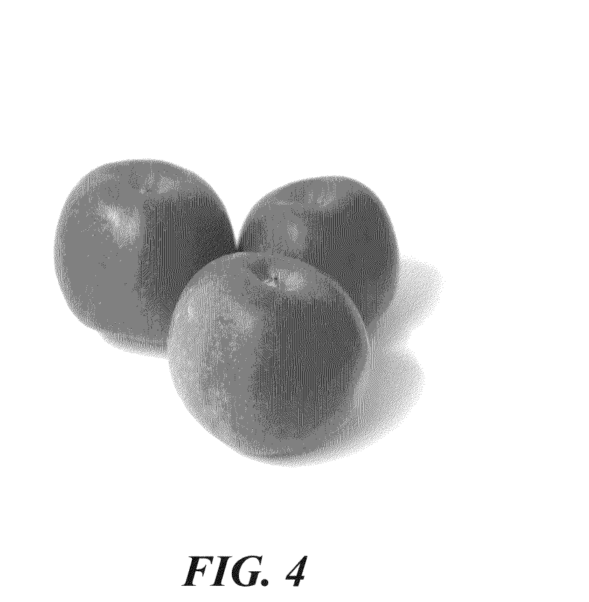

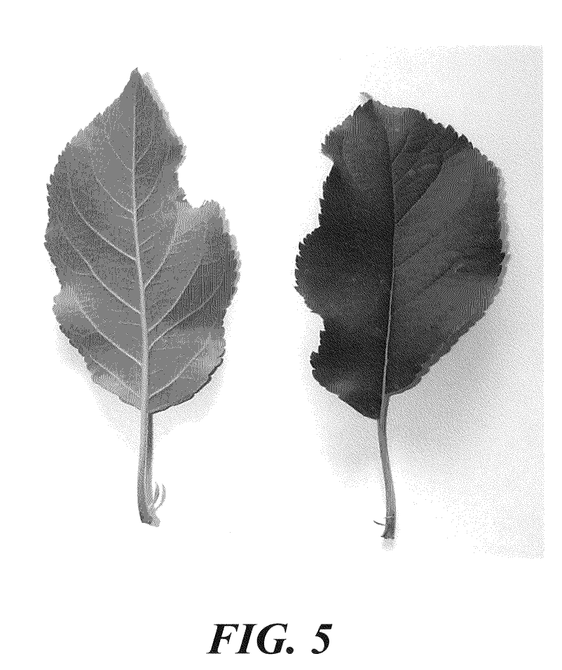

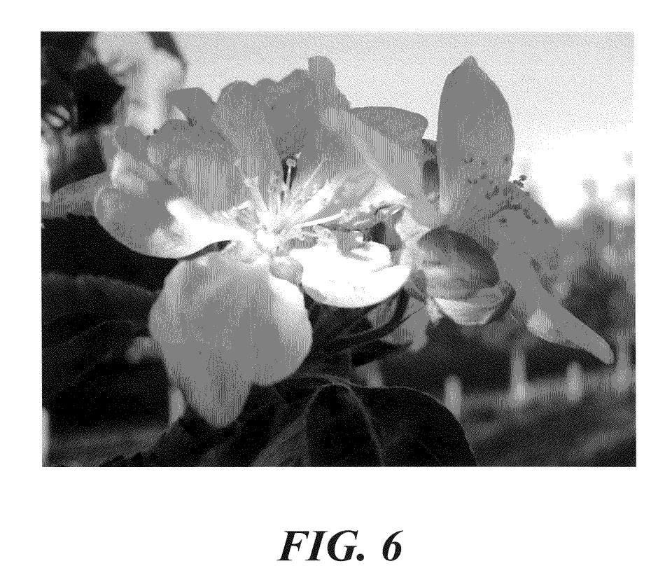

### DETAILED BOTANICAL DESCRIPTION

The following detailed botanical description is based on observations of 5^{th} generation trees planted in 2002, and described during the 2006-2007 and 2007-2008 growing seasons at Kirup, Western Australia. Color descriptions refer to The Royal Horticultural Society Colour Chart (1995). It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Medium to large with dense foliage, upright habit.
Vigor: Low; young trees average less than 80 cm of new growth during the growing season in the nursery row following bud placement.
Trunk: Medium stocky, diameter 6.4 cm at 30 cm above graft union; bark texture smooth; color greenish-brown 165B; Lenticels — size 2-3 mm, density 3-4/cm²; color brown 200A.
Branches: Thick, diameter 2.4 cm; crotch angle 70°; bark smooth; color greenish-brown 165B; Lenticels — size 1-2 mm, density 1-2/cm²; color brown 200A.
Chilling requirement: Low to moderate.
Dormant one year old shoot: Pubescence medium; diameter 10 mm; length 60-80 cm; color 165B; internode length 10 mm; Lenticels — size 1-2 mm, density 1-2/cm²; color brown 200A.
Flower buds: Quantity per spur 5; Shape round; Size — length 8-10 mm, diameter 8-10 mm; Color pink 57A.
Flowers: Size — Large, diameter 36-40 mm, depth 18-20 mm; Quantity per cluster 4-5; Petals — Quantity per flower 5; Margins free to touching, smooth; Length 18-20 mm; Width 14-15 mm; Apex shape round; Base shape round; Upper surface color white 55D with traces of pink 67A in veins and edges; Lower surface color white 155D with traces of pink 68A in veins and edges when fully open. Sepals — Quantity per flower 5; Shape pointed; Length 10 mm; Color green 135B. Pedicel — Length 20 mm; Diameter 2 mm; Color greyed purple 187. Pistil — Length 13-14 mm, color green 145B; Stigma — Size 1 mm, color yellow green 151A; Style — Length 8 mm, color green 145B; Ovary — Size 4-5 mm, color greyed purple 187. Stamen — Quantity per flower 18-22; Filament length 8 mm; Pollen color yellow 5C.
Bloom period: Late; In 2006, first bloom late October, full bloom 28 October; In 2007, first bloom late October, full bloom 4 November (Kirup, Western Australia).
Leaf: Attitude in relation to shoot outwards; Size — Length 10.5 cm, width 6 cm, length-width ratio 1.75; Shape oval, apex oval, convex, pointed, base round, margin biserrate; Color — Upper surface RHS 137B, lower surface RHS 147C; Petiole — Length 35-40 mm, diameter 2 mm, color RHS 141B.
Fruit: Size — Mass 190 g, diameter 77 mm, height 71 mm, ratio of height to width 0.93; Shape — General shape in profile Asymmetrical, globose to globose conical, position of maximum diameter middle, ribbing weak; Crowning at calyx end absent or weak; Eye — Aperture closed or slightly open, size medium, depth of eye basin 10 mm, width of eye basin 34 mm; Stalk — Diameter medium to thick, length medium, color greyed purple 185A, depth of stalk cavity 14-15 mm, width of stalk cavity 35 mm; Lenticels — Size small, quantity many; Skin — Bloom absent or weak, greasiness moderate, ground color yellow-green, overcolor pink-red, intensity of overcolor medium, percent of overcolor 80-100%, pattern of over color only solid flush; Flesh — Texture juicy, firm (8 kg pressure), color cream 155D; Seeds — Quantity per fruit 5-8, shape obtuse, color dark brown 200A; Aroma distinct, complex and highly aromatic; Yield 40 tonnes/hectare; Use dessert, market.
Harvest date: Late: 2007-May 3; 2008-May 13
Resistance/susceptibility to known diseases/pests: Unknown
Keeping quality: Good; over previous seasons fruit has stored and out-turned the same as ‘Cripps Pink.’

1. We claim a new and distinct apple tree substantially as shown and described herein.
